(19) United States
(12) Patent Application Publication
(10) Pub. No.: US 2006/0078528 A1
(43) Pub. Date: Apr. 13, 2006

(54) Hair conditioning composition comprising tight lamellar gel matrix

(75) Inventors: Jian-Zhong Yang, Kobe (JP); Koji Takata, Kobe (JP); Yujun Li, Kobe (JP); Hoyun Kim, Kobe (JP)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Compangy

(21) Appl. No.: 11/249,617

(22) Filed: Oct. 13, 2005

Related U.S. Application Data
(60) Provisional application No. 60/692,668, filed on Jun. 21, 2005.
(60) Provisional application No. 60/618,543, filed on Oct. 13, 2004.

(51) Int. Cl. A61K 8/41

(52) U.S. Cl. 424/70.27

(57) ABSTRACT

Disclosed is a hair conditioning composition comprising: a cationic surfactant; a high melting point fatty compound; and an aqueous carrier; wherein the cationic surfactant, the high melting point fatty compound, and the aqueous carrier form a lamellar gel matrix; wherein the d-spacing of the lamellar layers is in the range of 33 nm or less; and wherein the composition has a yield stress of about 30 Pa or more at 26.7° C. The composition of the present invention can provide improved conditioning benefits, especially improved slippery feel during the application to wet hair.

### CROSS REFERENCE TO RELATED APPLICATION

This application claims the benefit of U.S. Provisional Application No. 60/692,668, filed on Jun. 21, 2005 and U.S. Provisional Application No. 60/618,543 filed on Oct. 13, 2004.

### FIELD OF INVENTION

The present invention relates to a hair conditioning composition comprising: a cationic surfactant; a high melting point fatty compound; and an aqueous carrier; wherein the cationic surfactant, the high melting point fatty compound, and the aqueous carrier form a lamellar gel matrix; wherein the d-spacing of the lamellar layers is in the range of 33 nm or less; and wherein the composition has a yield stress of about 30 Pa or more at 26.7° C. The composition of the present invention can provide improved conditioning benefits, especially improved slippery feel during the application to wet hair.

### BACKGROUND OF THE INVENTION

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits. For example, some cationic surfactants, when used together with some high melting point fatty compounds and aqueous carrier, are believed to provide a gel matrix which is suitable for providing a variety of conditioning benefits such as slippery feel during the application to wet hair and softness and moisturized feel on dry hair. For example, WO 04/035016 discloses conditioning compositions comprising: a cationic crosslinked polymer; stearamidopropyl dimethylamine or behenyl trimethyl ammonium chloride; cetyl/stearyl alcohols; and water, in Examples. The conditioning compositions are said to provide improved conditioning benefits such as softness on wet substances, while providing slippery feel on wet substances and softness and moisturized feel on the substances when they are dried.

However, there remains a need for hair conditioning compositions which provide improved conditioning benefits, especially improved slippery feel during the application to wet hair.

Based on the foregoing, there remains a need for conditioning compositions which provide improved conditioning benefits, especially improved slippery feel during the application to wet hair.

None of the existing art provides all of the advantages and benefits of the present invention.

### SUMMARY OF THE INVENTION

The present invention is directed to a hair conditioning composition comprising by weight:
(a) from about 0.1% to about 10% of a cationic surfactant;
(b) from about 2.5% to about 15% by weight of the composition of a high melting point fatty compound; and
(c) and an aqueous carrier;
wherein the cationic surfactant, the high melting point fatty compound, and the aqueous carrier form a lamellar gel matrix; wherein the d-spacing of the lamellar layers is in the range of 33 nm or less; and wherein the composition has a yield stress of about 30 Pa or more at 26.7° C.

The conditioning composition of the present invention can provide improved conditioning benefits, especially improved slippery feel during the application to wet hair.

These and other features, aspects, and advantages of the present invention will become better understood from a reading of the following description, and appended claims.

### BRIEF DESCRIPTION OF THE FIGURE

While the specification concludes with claims particularly pointing out and distinctly claiming the invention, it is believed that the invention will be better understood from the following description of the accompanying figure in which:

FIG. 1 illustrates an embodiment of d-spacing measurement of the lamellar gel matrix.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

Composition

The present invention is directed to a hair conditioning composition comprising by weight:
(a) from about 0.1% to about 10% of a cationic surfactant;
(b) from about 2.5% to about 15% by weight of the composition of a high melting point fatty compound; and
(c) and an aqueous carrier;
wherein the cationic surfactant, the high melting point fatty compound, and the aqueous carrier form a lamellar gel matrix; wherein the d-spacing of the lamellar layers is in the range of 33 nm or less; and wherein the composition has a yield stress of about 30 Pa or more at 26.7° C.

The conditioning composition of the present invention can provide improved conditioning benefits, especially improved slippery feel during the application to wet hair.

It has been found that: hair conditioning compositions having a the above selected d-spacing and the above selected yield stress deliver improved wet conditioning benefits especially improved slippery feel during the application to wet hair, compared to compositions having a larger d-spacing and/or smaller yield stress. It is believed that compositions having such selected d-spacing and selected yield stress contain a larger amount of gel matrix containing a larger amount of lamellar gel matrix which results in tighter lamellar gel matrix, compared to compositions having a larger d-spacing and/or smaller yield stress. It has been found that the combination of selected d-spacing and selected yield stress can appropriately distinguish the compositions which deliver improved wet conditioning benefits, from other compositions.

Lamellar Gel Matrix

The composition of the present invention comprises a gel matrix including lamellar gel matrix, especially tighter lamellar gel matrix. The gel matrix comprises the cationic surfactant, the high melting point fatty compound, and an aqueous carrier. The gel matrix is suitable for providing various conditioning benefits such as slippery feel during the application to wet hair and softness and moisturized feel on dry hair. Among the gel matrix, lamellar gel matrix can provide improved slippery feel during the application to wet hair. Among the lamellar gel matrix, tighter lamellar gel matrix can provide improved slippery feel during the application to wet hair.

In view of improved wet conditioning benefits, the cationic surfactant and the high melting point fatty compound are contained at a level such that the weight ratio of the cationic surfactant to the high melting point fatty compound is in the range of, preferably from about 1:1 to 1:10, more preferably from about 1:1 to 1:4.

Preferably, the composition of the present invention comprises, by weight of the hair care composition, from about 60% to about 99%, preferably from about 70% to about 95%, and more preferably from about 80% to about 95% of a gel matrix including lamellar gel matrix, to which optional ingredients such as silicones can be added. The composition containing the above amount of gel matrix containing lamellar gel matrix is characterized by a yield stress of about 30 Pa or more, as measured by dynamic oscillation stress sweep at 1 Hz frequency and 26.7° C., by means of a rheometer available from TA Instruments with a mode name of AR2000 using 40 mm diameter parallel type geometry having gap of 1500 μm. Preferably, the composition of the present invention, especially for rinse-off use, has a yield stress of from about 30 Pa to about 90 Pa, more preferably from about 35 Pa to about 85 Pa, still more preferably from about 40 Pa to about 80 Pa, even more preferably from about 40 Pa to about 70 Pa.

The composition containing the above amount of gel matrix can be characterized by a shear stress at shear rate 950 s⁻¹ of from about 150 Pa to about 500 Pa, preferably from about 200 Pa to about 500 Pa, and more preferably from about 250 Pa to about 500 Pa, as measured at 26.7° C., by means of a rheometer available from TA Instruments with a mode name of AR2000 using 4 cm degree aluminum cone type geometry having gap of 57 μm. The composition containing the above amount of gel matrix can be characterized by a certain storage modulus, G′. Storage modulus, G′, is known as one of the widely used viscoelasticity parameters. G′ is defined as the part of the shear stress that is in phase with the shear strain divided by the strain under sinusoidal conditions. The composition containing the above amount of gel matrix is characterized by G′ of from about 2200 Pa to about 10000 Pa, preferably from about 2500 Pa to about 8000 Pa, as measured at 26.7° C. and 1 Hz frequency, by means of a rheometer available from TA Instruments with a mode name of AR2000 using 4 cm parallel type geometry having gap of 1500 μm. The composition containing the above amount of gel matrix can be characterized by its dilution profile. The compositions containing higher amount of gel matrix need a longer time to be homogenized with water when diluted. The composition containing the above amount of gel matrix can be characterized by measuring an amount of water which is not incorporated into the gel matrix.

The existence of lamellar gel matrix can be observed by cryo-scanning electronic microscopy (cryo-SEM). Preferably, the composition of the present invention has a higher amount of lamellar gel matrix. The amount of lamellar gel matrix can be measured by analyzing SEM picture, for example, by calculating area of lamellar gel matrix per unit area.

The existence of a gel matrix including a lamellar gel matrix may be detected by differential scanning calorimetry (hereinafter referred to as “DSC”) measurement of the composition. A profile chart obtained by DSC measurement describes chemical and physical changes of the scanned sample that involve an enthalpy change or energy gradient when the temperature of the sample is fluctuated. As such, the phase behavior and interaction among components of hair conditioning compositions of the present invention may be understood by their DSC profiles. DSC measurement of compositions of the present invention may be conducted by any suitable instrument available. For example, DSC measurement may be suitably conducted by Seiko DSC 6000 instrument available from Seiko Instruments Inc. In a typical measurement procedure, a sample is prepared by sealing an appropriate amount of the composition into a container made for DSC measurement and sealed. The weight of the sample is recorded. A blank sample i.e.; an unsealed sample of the same container is also prepared. The sample and blank sample are placed inside the instrument, and run under a measurement condition of from about −50° C. to about 130° C. at a heating rate of from about 1° C./minute to about 10° C./minute. The area of the peaks as identified are calculated and divided by the weight of the sample to obtain the enthalpy change in mJ/mg. The position of the peaks is identified by the peak top position.

In a preferred composition having a higher amount of gel matrix, the DSC profile shows a formation peak of larger than about 3 mJ/mg, more preferably from about 6 mJ/mg to about 10 mJ/mg. The DSC profile of a preferred composition shows a single peak having a peak top temperature of from about 55° C. to about 75° C. The DSC profile of the preferred composition shows no peaks larger than 3 mJ/mg, more preferably no peaks larger than 2.5 mJ/mg, still more preferably no peaks larger than 2 mJ/mg at a temperature of from 40° C. to 55° C., as the peaks showing at a temperature of from 40° C. to 55° C. mean the existence of high melting fatty compounds and/or cationic surfactants which are not incorporated into the gel matrix. It is believed that a composition formed predominantly with such a gel matrix shows a relatively stable phase behavior during the temperature range of from about 40° C. to about 55° C. In highly preferred composition, the DSC profile shows a single peak having a peak top temperature of about 67° C. to about 73° C., at about 8 mJ/mg, and no peaks larger than 2 mJ/mg from 40° C. to about 65° C.

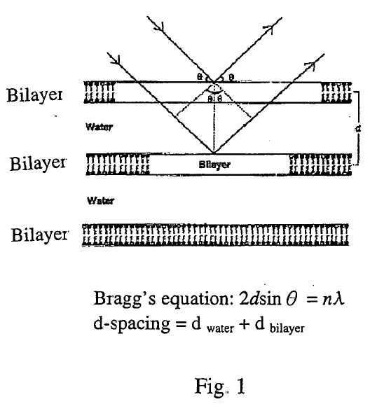

The existence of a lamellar gel matrix is also detected by d-spacing. The compositions of the present invention have d-spacing value of 33 nm or less, preferably 31 nm or less, more preferably 28 nm or less. D-spacing in the present invention means a distance between two lamellar bilayers plus the width of one lamellar bilayer, as shown in FIG. 1. Thus, d-spacing is defined according to the following equation:
D-spacing=d_{water}+d_{bilayer}

D-spacing can be measured by using a High Flux Small Angle X-ray Scattering Instrument available from PANalytical with a tradename SAXSess, under the typical conditions of Small Angle X-Ray Scattering (SAXS) measurements in a q-range (q=4π/λ sin(θ) wherein λ is the wavelength and θ is half the scattering angel) of 0.06<q/nm⁻¹<27 which corresponds to 0.085<2θ/degree<40. All data are transmission-calibrated by monitoring the attenuated primary beam intensity and normalizing it into unity, so that relative intensity for different samples can be obtained. The transmission-calibration allows us to make an accurate subtraction of water contribution from the net sample scattering. D-spacing is calculated according to the following equation (which is known as Bragg's equation):
nλ=2d sin(θ), wherein n is the number of lamellar bi-layers

It has been found that: hair conditioning compositions having a smaller d-spacing (i.e., tighter sheet-like lamellar gel matrix) and the above selected yield stress deliver improved wet conditioning benefits especially improved slippery feel during the application to wet hair, compared to compositions having a larger d-spacing and/or smaller yield stress. It is believed that compositions having such selected d-spacing and selected yield stress contain a larger amount of gel matrix containing a larger amount of sheet-like lamellar gel matrix, compared to compositions having a larger d-spacing and/or smaller yield stress. It has been found that the combination of selected d-spacing and selected yield stress can appropriately distinguish the compositions which deliver improved wet conditioning benefits, from other compositions. In addition to the above selected d-spacing and selected yield stress, the above preferred amount of total gel matrix, and/or preferred amount of lamellar gel matrix further help the composition of the present invention be distinguished from other compositions.

It has been also found that: preferred cationic surfactants of the present invention can provide improved wet conditioning benefits, compared to other cationic surfactants such as tertiary amine, tertiary amine salt, and di-long alkyl quaternized ammonium salt. Thus, it is preferred in the present invention that, in view of providing improved wet conditioning benefits, the composition is substantially free of other cationic surfactants than those preferred in the present invention. Such “other cationic surfactant” includes, for example, mono-long alkyl quaternized ammonium salt in which the anion is not C1-C4 alkyl sulfate, tertiary amines, tertiary amine salts, and di-long alkyl quaternized ammonium salts. In the present invention, “substantially free of other cationic surfactants” means that the composition contain 1% or less, preferably 0.5% or less, more preferably totally 0% of total of such other cationic surfactants.

Preferably, in view of stability of the gel matrix, the composition of the present invention is substantially free of anionic surfactants and anionic polymers. In the present invention, “substantially free of anionic surfactants and anionic polymers” means that the composition contains 1% or less, preferably 0.5% or less, more preferably totally 0% of total of anionic surfactants and anionic polymers.

In view of improved wet conditioning benefits, it is preferred to contain the cationic surfactant and the high melting point fatty compound at a level such that the weight ratio of the cationic surfactant to the high melting point fatty compound is in the range of from about 1:1 to 1:10, more preferably from about 1:1 to 1:4. In view of improved wet conditioning benefits, especially when containing mono-long alkyl quaternized ammonium salts as a cationic surfactant, it is also preferred to contain the mono-long alkyl quaternized ammonium salt and the high melting point fatty compound at a level such that the total amount of the mono-long alkyl quaternized ammonium salt and the high melting point fatty compound is 5% or more, more preferably 6.5% or more, and still more preferably 7.5% or more by weight of the composition.

For forming gel matrix including lamellar gel matrix, it is preferred to prepare the composition by the following method:

Water is typically heated to at least about 70° C., preferably between about 80° C. and about 90° C. The cationic surfactant and the high melting point fatty compound are combined with the water to form a mixture. The temperature of the mixture is preferably maintained at a temperature higher than both the melting temperature of the cationic surfactant and the melting temperature of the high melting point fatty compound, and the entire mixture is homogenized. After mixing until no solids are observed, the mixture is gradually cooled (e.g., at a rate of from about 1° C./minute to about 5° C./minute) to a temperature below 60° C., preferably less than about 55° C. During this gradual cooling process, a significant viscosity increase is observed at between about 55° C. and about 75° C. This indicates the formation of gel matrix including lamellar gel matrix. Additional components are then combined with the gel matrix, and cooled to room temperature.

Cationic Surfactant

The compositions of the present invention comprise a cationic surfactant. Among a variety of cationic surfactants, preferred are: (i) a salt of a mono-long alkyl quaternized ammonium and an anion, wherein the anion is selected from the group consisting of C1-C4 alkyl sulfate such as methosulfate and ethosulfate, and mixtures thereof; and (ii) an alkyl diquaternized ammonium salt. The cationic surfactant is included in the composition at a level by weight of from about 0.1% to about 10%. When containing mono-long alkyl quaternized ammonium salts as cationic surfactants, the mono-long alkyl quaternized ammonium salts are contained at a level by weight of preferably from about 1% to about 8%, more preferably from about 2% to about 5%, in view of improved wet conditioning benefits. When containing alkyl diquaternized ammonium salts as cationic surfactants, the alkyl diquaternized ammonium salts are contained at a level by weight of preferably from about 0.5% to about 5%, more preferably from about 0.8% to about 3%, in view of improved wet conditioning benefits especially for rinse-off use.

It is preferred in the present invention that, in view of improved wet conditioning benefits, the composition is substantially free of other cationic surfactants than those preferred in the present invention. Such “other cationic surfactant” includes, for example, mono-long alkyl quaternized ammonium salt in which the anion is not C1-C4 alkyl sulfate, tertiary amines, tertiary amine salts, and di-long alkyl quaternized ammonium salts. In the present invention, “substantially free of other cationic surfactants” means that the composition contains 1% or less, preferably 0.5% or less, more preferably totally 0% of total of such other cationic surfactants.

(i) Mono-Long Alkyl Quaternized Ammonium Salt Cationic Surfactant

One of the preferred cationic surfactants of the present invention is a salt of a mono-long alkyl quaternized ammonium and an anion, wherein the anion is selected from the group consisting of C1-C4alkyl sulfate such as methosulfate and ethosulfate, and mixtures thereof. It has been surprisingly found that: by the use of the selected anions having more ion binding strength compared to other anions such as chloride, the cationic surfactants have reduced hydrated radius; such reduced hydrated radius results in tighter lamellar gel matrix, i.e., reduced distance between one lamellar bilayer and another lamellar bilayer.

The mono-long alkyl quaternized ammonium salts useful herein are those having the formula (I): 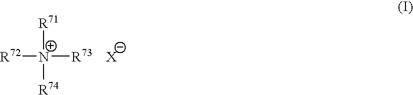
wherein one of R⁷¹, R⁷², R⁷³ and R⁷⁴ is selected from an aliphatic group of from 16 to 40 carbon atoms or an aromatic, alkoxy, polyoxyalkylene, alkylamido, hydroxyalkyl, aryl or alkylaryl group having up to about 40 carbon atoms; the remainder of R⁷¹, R⁷², R⁷³ and R⁷⁴ are independently selected from an aliphatic group of from 1 to about 8 carbon atoms or an aromatic, alkoxy, polyoxyalkylene, alkylamido, hydroxyalkyl, aryl or alkylaryl group having up to about 8 carbon atoms; and X⁻ is a salt-forming anion selected from the group consisting of C1-C4 alkyl sulfate such as methosulfate and ethosulfate and mixtures thereof. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Preferably, one of R⁷¹, R⁷², R⁷³ and R⁷⁴ is selected from an alkyl group of from 16 to 40 carbon atoms, more preferably from 18 to 26 carbon atoms, still more preferably from 22 carbon atoms; and the remainder of R⁷¹, R⁷², R⁷³ and R⁷⁴ are independently selected from CH₃, C₂H₅, C₂H₄₀H, CH₂C₆H₅, and mixtures thereof. It is believed that such mono-long alkyl quaternized ammonium salts can provide improved slippery and slick feel on wet hair, compared to multi-long alkyl quaternized ammonium salts. It is also believed that mono-long alkyl quaternized ammonium salts can provide improved hydrophobicity and smooth feel on dry hair, compared to amine or amine salt cationic surfactants.

Among them, more preferred cationic surfactants are those having a longer alkyl group, i.e., C18-22 alkyl group. Such cationic surfactants include, for example, behenyl trimethyl ammonium methyl sulfate or ethyl sulfate and stearyl trimethyl ammonium methyl sulfate or ethyl sulfate, and still more preferred is behenyl trimethyl ammonium methyl sulfate or ethyl sulfate. It is believed that; cationic surfactants having a longer alkyl group provide improved deposition on the hair, thus can provide improved conditioning benefits such as improved softness on dry hair, compared to cationic surfactant having a shorter alkyl group. It is also believed that such cationic surfactants can provide reduced irritation, compared to cationic surfactants having a shorter alkyl group.

(ii) Alkyl Diquaternized Ammonium Salt Cationic Surfactant

The other cationic surfactants which are preferred in the present invention are an alkyl diquaternized ammonium salt. The alkyl diquaternized ammonium salt cationic surfactants useful herein are those having two quaternized nitrogen atoms. It has been surprisingly found that: by having two quaternized nitrogen atoms, the alkyl diquaternized ammonium salt results in tighter lamellar gel matrix, i.e., reduced distance between one lamellar phase and another lamellar phase, compared to other cationic surfactants such as behentrimethylammonium chloride and stearylamidopropyldimethylamine.

The alkyl diquaternized ammonium salt cationic surfactants useful herein are those selected from the group consisting of following (A1), (A2), and mixtures thereof:
(A1) an alkyl diquaternized ammonium salt having the formula: 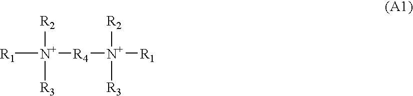
   wherein R₁ represents straight or branched, saturated or unsaturated, and/or functionalized or non-functionalized C12-C40 alkyl chain, wherein the functionalized alkyl chain is that containing one or more functional groups selected from the group consisting of —OH, —COO—, —OCO—, —CONH—, —NHCO—, —O—, and —(R₅—O)ₙ— wherein R₅ is C2-4 alkylene and n is integer from 0 to 30; R₂ represents C1-3 alkyl, hydroxyalkyl, or —(R₅—O)ₙ—H; R₃ represents R₂ or H; and R₄ represents functionalized or non-functionalized C2-8 alkylene, wherein the functionalized alkylene is that containing one or more functional groups selected from the group consisting of —OH, —COO—, —OCO—, —CO—NH—, —O—, and —(R₅—O)ₙ—; and
(A2) an alkyl diquaternized ammonium salt having the formula: 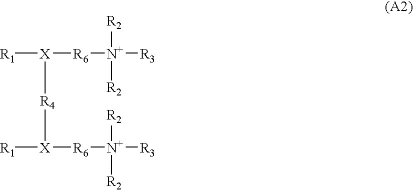
   wherein R₁-R₄ represent the same structures as described above; X represents CH or N; and R₆ represents functionalized or non-functionalized C1-10 alkylene wherein the functionalized alkylene is that containing one or more functional groups selected from the group consisting of —OH, —COO—, —OCO—, —CONH—, —NHCO—, —O—, and —(R₅—O)ₙ—.

Preferably, the alkyl diquaternized ammonium salt cationic surfactants are selected from the group consisting of: those having the formula (A1) wherein R₄ is C2-8 alkylene functionalized with a —OH group such as those having an INCI name “Hydroxypropyl-bis-Stearyl-N,N-Dimethylammonium Chloride)”; those having the formula (A2); and mixtures thereof.

High Melting Point Fatty Compound

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols. Preferred fatty alcohols include, for example, cetyl alcohol, stearyl alcohol, behenyl alcohol, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

Commercially available high melting point fatty compounds useful herein include: cetyl alcohol, stearyl alcohol, and behenyl alcohol having tradenames KONOL series available from Shin Nihon Rika (Osaka, Japan), and NAA series available from NOF (Tokyo, Japan); pure behenyl alcohol having tradename 1-DOCOSANOL available from WAKO (Osaka, Japan).

The high melting point fatty compound is included in the composition at a level of from about 5% to about 15%, preferably from about 5.5% to about 10%, more preferably from about 6% to about 8% by weight of the composition, in view of providing improved wet conditioning benefits.

Aqueous Carrier

The conditioning composition of the present invention comprises an aqueous carrier. The level and species of the carrier are selected according to the compatibility with other components, and other desired characteristic of the product.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 30% to about 95%, and more preferably from about 80% to about 90% water.

Silicone Compound

Preferably, the compositions of the present invention preferably contain a silicone compound. It is believed that the silicone compound can provide smoothness and softness on dry hair. The silicone compounds herein can be used at levels by weight of the composition of preferably from about 0.1% to about 20%, more preferably from about 0.5% to about 10%, still more preferably from about 1% to about 8%.

The silicone compounds useful herein, as a single compound, as a blend or mixture of at least two silicone compounds, or as a blend or mixture of at least one silicone compound and at least one solvent, have a viscosity of preferably from about 1,000 to about 2,000,000 mPa·s at 25° C.

The viscosity can be measured by means of a glass capillary viscometer as set forth in Dow Corning Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used.

Preferably, the silicone compounds have an average particle size of from about 1 microns to about 50 microns, in the composition.

The silicone compounds useful herein include polyalkyl or polyaryl siloxanes with the following structure: 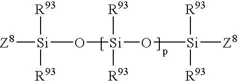
wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil® and TSF 451 series, and from Dow Corning in their Dow Corning SH200 series.

The above polyalkylsiloxanes are available, for example, as a mixture with silicone compounds having a lower viscosity. Such mixtures have a viscosity of preferably from about 1,000 mPa·s to about 100,000 mPa·s, more preferably from about 5,000 mPa·s to about 50,000 mPa·s. Such mixtures preferably comprise: (i) a first silicone having a viscosity of from about 100,000 mPa·s to about 30,000,000 mPa·s at 25° C., preferably from about 100,000 mPa·s to about 20,000,000 mPa·s; and (ii) a second silicone having a viscosity of from about 5 mPa·s to about 10,000 mPa·s at 25° C., preferably from about 5 mPa·s to about 5,000 mPa·s. Such mixtures useful herein include, for example, a blend of dimethicone having a viscosity of 18,000,000 mPa·s and dimethicone having a viscosity of 200 mPa·s available from GE Toshiba, and a blend of dimethicone having a viscosity of 18,000,000 mPa·s and cyclopentasiloxane available from GE Toshiba.

The silicone compounds useful herein also include a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. The silicone gums are available, for example, as a mixture with silicone compounds having a lower viscosity. Such mixtures useful herein include, for example, Gum/Cyclomethicone blend available from Shin-Etsu.

The silicone compounds that can be used include, for example, a polypropylene oxide modified polydimethylsiloxane although ethylene oxide or mixtures of ethylene oxide and propylene oxide can also be used. The ethylene oxide and polypropylene oxide level should be sufficiently low so as not to interfere with the dispersibility characteristics of the silicone. These materials are also known as dimethicone copolyols.

Silicone compounds useful herein also include amino substituted materials. Preferred aminosilicones include, for example, those which conform to the general formula (I):
(R₁)ₐG_{3-α}-Si—(—OSiG₂)ₙ-(OSiG_{b}(R₁)_{2-b})ₘ—O—SiG₃₋ₐ(R₁)ₐ
wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion.

Highly preferred amino silicones are those corresponding to formula (I) wherein m=0, a=1, q=3, G=methyl, n is preferably from about 1500 to about 1700, more preferably about 1600; and L is —N(CH₃)₂ or —NH₂, more preferably —NH₂. Another highly preferred amino silicones are those corresponding to formula (1) wherein m=0, a=1, q=3, G=methyl, n is preferably from about 400 to about 600, more preferably about 500; and L is —N(CH₃)₂ or —NH₂, more preferably —NH₂. Such highly preferred amino silicones can be called as terminal aminosilicones, as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

Other suitable alkylamino substituted silicone compounds include those represented by the following structure: 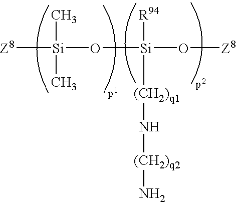
wherein R⁹⁴ is H, CH₃ or OH; p¹ and p² are integers of 1 or above, and wherein sum of p¹ and p² is from 650 to 1,500; q¹ and q² are integers of from 1 to 10. Z⁸ represents groups which block the ends of the silicone chains. Suitable Z⁸ groups include hydroxy, methyl, methoxy, ethoxy, propoxy, and aryloxy. Highly preferred are those known as “amodimethicone”. Commercially available amodimethicones useful herein include, for example, BY16-872 available from Dow Corning.

Other amino substituted silicone polymers which can be used are represented by the formula:

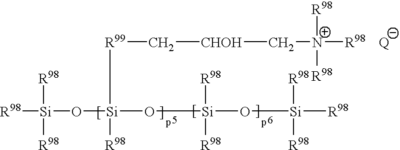

where R⁹⁸ denotes a monovalent hydrocarbon radical having from 1 to 18 carbon atoms, preferably an alkyl or alkenyl radical such as methyl; R⁹⁹ denotes a hydrocarbon radical, preferably a C₁-C₁₈ alkylene radical or a C₁-C₁₈, and more preferably C₁-C₈, alkyleneoxy radical; Q⁻ is a halide ion, preferably chloride; p⁵ denotes an average statistical value from 2 to 20, preferably from 2 to 8; p⁶ denotes an average statistical value from 20 to 200, and preferably from 20 to 50.

The silicone compounds may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof.

Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; coloring agents, such as any of the FD&C or D&C dyes; perfumes; and sequestering agents, such as disodium ethylenediamine tetra-acetate; ultraviolet and infrared screening and absorbing agents such as benzophenones; and antidandruff agents such as zinc pyrithione.

Low Melting Point Oil

Low melting point oils useful herein are those having a melting point of less than 25° C. The low melting point oil useful herein is selected from the group consisting of: hydrocarbon having from 10 to about 40 carbon atoms; unsaturated fatty alcohols having from about 10 to about 30 carbon atoms such as oleyl alcohol; unsaturated fatty acids having from about 10 to about 30 carbon atoms; fatty acid derivatives; fatty alcohol derivatives; ester oils such as pentaerythritol ester oils, trimethylol ester oils, citrate ester oils, and glyceryl ester oils; poly α-olefin oils; and mixtures thereof. Preferred low melting point oils herein are selected from the group consisting of: ester oils such as pentaerythritol ester oils, trimethylol ester oils, citrate ester oils, and glyceryl ester oils; poly α-olefin oils; and mixtures thereof,

Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof. Such compounds are available from Kokyo Alcohol with tradenames KAKPTI, KAKTTI, and Shin-nihon Rika with tradenames PTO, ENUJERUBU TP3SO.

Particularly useful citrate ester oils herein include triisocetyl citrate with tradename CITMOL 316 available from Bernel, triisostearyl citrate with tradename PELEMOL TISC available from Phoenix, and trioctyldodecyl citrate with tradename CITMOL 320 available from Bernel.

Particularly useful glyceryl ester oils herein include triisostearin with tradename SUN ESPOL G-318 available from Taiyo Kagaku, triolein with tradename CITHROL GTO available from Croda Surfactants Ltd., trilinolein with tradename EFADERMA-F available from Vevy, or tradename EFA-GLYCERIDES from Brooks.

Particularly useful poly α-olefin oils herein include polydecenes with tradenames PURESYN 6 having a number average molecular weight of about 500 and PURESYN 100 having a number average molecular weight of about 3000 and PURESYN 300 having a number average molecular weight of about 6000 available from Exxon Mobil Co.

Product Forms

The conditioning compositions of the present invention can be in the form of rinse-off products or leave-on products, and can be formulated in a wide variety of product forms, including but not limited to creams, gels, emulsions, mousses and sprays. The conditioning composition of the present invention is especially suitable for rinse-off hair conditioner.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

Compositions (wt %)

| Components | Ex. 1 | Ex. 2 | Ex. 3 | Ex. 4 | Ex. 5 | Ex. 6 | Ex. 7 | Ex. 8 |
|---|---|---|---|---|---|---|---|---|
| Behenyl trimethyl ammonium | 2.25 | 1.76 | 1.98 | 2.70 | 3.38 | — | 1.20 | 1.80 |
| methyl sulfate | | | | | | | | |
| Stearyl trimethyl ammonium | — | — | — | — | — | 2.50 | 1.30 | 1.20 |
| ethyl sulfate | | | | | | | | |
| Cetyl alcohol | 1.88 | 1.45 | 1.65 | 2.26 | 2.32 | 2.50 | 2.50 | 2.50 |
| Stearyl alcohol | 4.64 | 3.62 | 4.08 | 5.57 | 4.18 | 4.50 | 3.50 | 4.50 |
| Behenyl alcohol | — | — | — | — | — | — | 1.00 | 0.50 |
| Dimethicone blend *1 | — | — | — | — | — | 4.20 | — | — |
| Dimethicone/Cyclomethicone *2 | — | — | — | — | — | — | 4.20 | — |
| Aminosilicone-1 *3 | 3.50 | 3.50 | — | 3.00 | — | — | — | 2.00 |
| Aminosilicone-2 *4 | — | — | 3.50 | — | 3.50 | — | — | — |
| Isoparaffins *5 | — | — | 1.50 | — | 1.50 | — | — | — |
| Isopropanol | 0.28 | 0.22 | 0.25 | 0.34 | 0.42 | — | — | 0.50 |
| Disodium EDTA | 0.13 | 0.13 | 0.13 | 0.13 | 0.13 | 0.13 | 0.13 | 0.13 |
| Sodium hydroxide | — | — | — | 0.010 | 0.014 | — | — | — |
| Benzyl alcohol | 0.4 | 0.4 | 0.4 | 0.4 | 0.4 | 0.4 | 0.4 | 0.4 |
| Kathon CG *6 | 0.033 | 0.033 | 0.033 | 0.033 | 0.033 | 0.033 | 0.033 | 0.033 |
| Perfume | 0.35 | 0.35 | 0.25 | 0.25 | 0.25 | 0.25 | 0.25 | 0.25 |
| Panthenol | 0.05 | 0.05 | 0.05 | — | — | — | 0.05 | 005 |
| Panthenyl ethyl ether | 0.05 | 0.05 | 0.05 | — | — | — | 0.05 | 0.05 |
| Vitamin E | — | — | — | — | 0.01 | 0.01 | 0.01 | 0.01 |
| Octyl methoxycinnamate | — | — | — | — | — | — | 0.09 | 0.09 |
| Benzophenone-3 | — | — | — | — | — | — | 0.09 | 0.09 |

| Deionized Water | q.s. to 100% |
|---|---|
| Definitions of Components | |
| *1 Dimethicone blend: a blend of dimethicone having a viscosity of 18,000,000 mPa · s and dimethicone having a viscosity of 200 mPa · s available from GE Toshiba | |
| *2 Dimethicone/Cyclomethicone: a blend dimethicone having a viscosity of 18,000,000 mPa · s and cyclopentasiloxane available from GE Toshiba | |
| *3 Aminosilicone-1: Available from GE having a viscosity 10,000 mPa · s, and having following formula (I): (R₁)ₐG₃₋ₐ—Si—(—OSiG₂)ₙ—(—OSiG_{b}(R₁)_{2-b})ₘ—O—SiG₃₋ₐ(R₁)ₐ (I) wherein G is methyl; a is an integer of 1; b is 0, 1 or 2, preferably 1; n is a number from 400 to about 600; m is an integer of 0; R₁ is a monovalent radical | |
| # conforming to the general formula CqH_{2q}L, wherein q is an integer of 3 and L is - NH₂ | |
| *4 Aminosilicone-2: Available from GE under trade name BX3083-1, has a viscosity range from 220,000-245,000 mPa · s, and having following formula (I): (R₁)ₐG₃₋ₐ—Si—(—OSiG₂)ₙ—(—OSiG_{b}(R₁)_{2-b})ₘ—O—SiG₃₋ₐ(R₁)ₐ (I) wherein G is methyl; a is an integer of 1; b is 0, 1 or 2, preferably 1; n is a number from 1500 to about 1700; m is an integer of 0; R₁ is a monovalent radical | |
| # conforming to the general formula CqH_{2q}L, wherein q is an integer of 3 and L is - NH₂ | |

*5 Isosol 400 available from NISSEKI
*6 Kathon CG: Available from Rohm&Haas

Compositions (wt %)

| Components | Ex. 9 | Ex. 10 | Ex. 11 | Ex. 12 | Ex. 13 |
|---|---|---|---|---|---|
| Alkyl diquaternized ammonium salt-1 *1 | 2.25 | 1.80 | 1.98 | 1.13 | 1.69 |
| Cetyl alcohol | 1.86 | 1.50 | 1.65 | 0.93 | 1.16 |
| Stearyl alcohol | 4.64 | 2.40 | 4.08 | 2.32 | 2.09 |
| Silicone compound-1 *5 | — | 4.2 | — | — | — |
| Silicone compound-2 *6 | — | — | 4.2 | — | — |
| Silicone compound-3 *7 | — | — | 3.50 | — | 3.50 |
| Silicone compound-4 *8 | 3.50 | — | — | 3.00 | — |
| Isoparaffins *9 | — | — | 1.50 | — | 1.50 |
| Benzyl alcohol | 0.4 | 0.4 | 0.4 | 0.4 | 0.4 |
| Methylchloroisothiazolinone/ | 0.0005 | 0.0005 | 0.0005 | 0.0005 | 0.0005 |
| Methylisothiazolinone *10 | | | | | |
| Perfume | 0.5 | 0.5 | 0.5 | 0.5 | 0.5 |
| NaOH | — | 0.014 | 0.014 | 0.014 | 0.014 |
| Panthenol | — | 0.05 | 0.05 | 0.05 | — |
| Panthenyl ethyl ether | — | 0.05 | 0.05 | 0.05 | — |
| Hydrolyzed collagen | — | — | 0.01 | 0.01 | 0.01 |
| Vitamin E | — | — | 0.01 | 0.01 | 0.01 |
| Octyl methoxycinnamate | — | — | — | 0.09 | — |
| Benzophenone-3 | — | — | — | 0.09 | — |
| Disodium EDTA | 0.127 | 0.127 | 0.127 | 0.127 | 0.127 |
| Deionized Water | q.s. to 100% | | | | |
| Components | Ex. 14 | Ex. 15 | Ex. 16 | Ex. 17 | Ex. 18 |
| Alkyl diquaternized ammonium salt-1 *1 | — | — | — | — | 0.00 |
| Alkyl diquaternized ammonium salt-2 *2 | 2.50 | 1.69 | — | — | 1.00 |
| Alkyl diquaternized ammonium salt-3 *3 | — | — | 1.69 | — | — |
| Alkyl diquaternized ammonium salt-4 *4 | — | — | — | 1.69 | — |
| Cetyl alcohol | 2.50 | 1.16 | 1.16 | 1.16 | 1.16 |
| Stearyl alcohol | 4.50 | 2.09 | 2.09 | 2.09 | 2.09 |
| Silicone compound-1 *5 | — | — | — | — | 1.0 |
| Silicone compound-2 *6 | — | — | — | 0.5 | — |
| Silicone compound-3 *7 | — | — | 3.5 | 1.0 | 3.5 |
| Silicone compound-4 *8 | 3.50 | 3.50 | — | 3.00 | — |
| Isoparaffins *9 | — | — | 1.50 | — | 1.50 |
| Benzyl alcohol | 0.4 | 0.4 | 0.4 | 0.4 | 0.4 |
| Methylchloroisothiazolinone/ | 0.0005 | 0.0005 | 0.0005 | 0.0005 | 0.0005 |
| Methylisothiazolinone *10 | | | | | |
| Perfume | 0.5 | 0.5 | 0.5 | 0.5 | 0.5 |
| NaOH | 0.014 | 0.014 | 0.014 | 0.014 | — |
| Panthenol | 0.05 | 0.05 | 0.05 | 0.05 | — |
| Panthenyl ethyl ether | 0.05 | 0.05 | 0.05 | 0.05 | — |
| Hydrolyzed collagen | — | 0.01 | — | — | — |
| Vitamin E | — | 0.01 | — | — | — |
| Octyl methoxycinnamate | — | 0.09 | — | — | — |
| Benzophenone-3 | — | 0.09 | — | — | — |
| Disodium EDTA | 0.127 | 0.127 | 0.127 | 0.127 | 0.127 |

| Deionized Water | q.s. to 100% |
|---|---|
| Definitions of Components | |
| *1 Alkyl diquaternized ammonium salt-1: Hydroxypropyl-bis-stearyl-N,N-dimethylammonium chloride available from Toho Kagaku K.K. | |
| *2 Alkyl diquaternized ammonium salt-2: Hydroxypropyl-bis-lauryl-N,N-dimethylammonium chloride available from Toho Kagaku K.K. | |
| *3 Alkyl diquaternized ammonium salt-3: Hydroxypropyl-bis-stearamidopropyl-N,N-dimethylammonium chloride available from Toho Kagaku K.K. | |
| *4 Alkyl diquaternized ammonium salt-4: Hydroxypropyl-bis-behenyl-N,N-dimethylammonium chloride available from Toho Kagaku K.K. | |
| *5 Silicone compound-1: Dimethicone/Cyclomethicone: a blend dimethicone having a viscosity of 18,000,000 mPa · s and cyclopentasiloxane available from GE Toshiba | |
| *6 Silicone compound-2: Dimethicone blend: a blend of dimethicone having a viscosity of 18,000,000 mPa · s and dimethicone having a viscosity of 200 mPa · s available from GE Toshiba | |
| *7 Silicone compound-3: Terminal aminosilicone which is available from GE under trade name BX3083-1, has a viscosity range from 220,000-245,000 mPa · s, and having following formula (I): (R₁)ₐG₃₋ₐ—Si—(—OSiG₂)ₙ—(—OSiG_{b}(R₁)_{2-b})ₘ—O—SiG₃₋ₐ(R₁)ₐ (I) wherein G is methyl; a is an integer of 1; b is 0, 1 or 2, preferably 1; n is a number | |
| # from 1500 to about 1700; m is an integer of 0; R₁ is a monovalent radical conforming to the general formula CqH_{2q}L, wherein q is an integer of 3 and L is - N(CH₃)₂ | |
| *8 Silicone compound-4: Available from GE having a viscosity 10,000 mPa · s, and having following formula (I): (R₁)ₐG₃₋ₐ—Si—(—OSiG₂)ₙ—(—OSiG_{b}(R₁)_{2-b})ₘ—O—SiG₃₋ₐ(R₁)ₐ (I) wherein G is methyl; a is an integer of 1; b is 0, 1 or 2, preferably 1; n is a number from 400 to about 600; m is an integer of 0; R₁ is a monovalent radical | |
| # conforming to the general formula CqH_{2q}L, wherein q is an integer of 3 and L is - N(CH₃)₂ | |

*9 Isosol 400 available from NISSEKI
*10 Methylchloroisothiazolinone/Methylisothiazolinone: Kathon CG available from Rohm & Haas

Method of Preparation

The conditioning compositions of “Ex. 1” through “Ex. 18” as shown above can be prepared by any conventional method well known in the art. They are suitably made as follows:

Cationic surfactants and high melting point fatty compounds are added to water with agitation, and heated to about 80° C. The mixture is cooled down to about 55° C. If included, silicone compounds, perfumes, preservatives are added to the mixture with agitation. Then the mixture is cooled down to room temperature.

Examples 1 through 18 are hair conditioning compositions of the present invention which are particularly useful for rinse-off use. The composition of Example 5 has a d-spacing value of 22 nm, a yield stress of 45 Pa at 26.7° C., and a shear stress at shear rate 950 s⁻¹ of 350 Pa at 26.7° C. The composition of Example 9 has a d-spacing value of 26 nm, a yield stress of 80 Pa at 26.7° C. The embodiments disclosed and represented by the previous “Ex. 1” through “Ex. 18” have many advantages. For example, they can provide improved wet conditioning benefits such as improved slippery feel on wet hair during the application, while maintaining improved dry conditioning benefits such as softness and moisturized feel on dry hair.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair conditioning composition comprising by weight:
(a) from about 0.1% to about 10% of a cationic surfactant;
(b) from about 2.5% to about 15% by weight of the composition of a high melting point fatty compound; and
(c) and an aqueous carrier;
wherein the cationic surfactant, the high melting point fatty compound, and the aqueous carrier form a lamellar gel matrix; wherein the d-spacing of the lamellar layers is in the range of 33 nm or less; and wherein the composition has a yield stress of about 30 Pa or more at 26.7° C.

2. The hair conditioning composition of claim 1 wherein the d-spacing is 31 nm or less.

3. The hair conditioning composition of claim 1 wherein the d-spacing is 28 nm or less.

4. The hair conditioning composition of claim 1 wherein the composition has a yield stress of from about 0.30 Pa to about 90 Pa.

5. The hair conditioning composition of claim 1 wherein the composition has a yield stress of from about 35 Pa to about 85 Pa.

6. The hair conditioning composition of claim 1 wherein the composition has a yield stress of from about 40 Pa to about 80 Pa.

7. The hair conditioning composition of claim 1 wherein the composition has a yield stress of from about 40 Pa to about 70 Pa.

8. The hair conditioning composition of claim 1 wherein the cationic surfactant is a salt of a mono-long alkyl quaternized ammonium and an anion, wherein the anion is selected from the group consisting of C1-C4 alkyl sulfate, and mixtures thereof.

9. The hair conditioning composition of claim 1 wherein the salt of a mono-long alkyl quaternized ammonium and an anion has the formula (I): 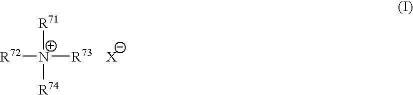
wherein one of R⁷¹, R⁷², R⁷³ and R⁷⁴ is selected from an aliphatic group of from 16 to 30 carbon atoms or an aromatic, alkoxy, polyoxyalkylene, alkylamido, hydroxyalkyl, aryl or alkylaryl group having up to about 30 carbon atoms; the remainder of R⁷¹, R⁷², R⁷³ and R⁷⁴ are independently selected from an aliphatic group of from 1 to about 8 carbon atoms or an aromatic, alkoxy, polyoxyalkylene, alkylamido, hydroxyalkyl, aryl or alkylaryl group having up to about 8 carbon atoms; and X⁻ is a salt-forming anion selected from the group consisting of C1-C4 alkyl sulfate and mixtures thereof.

10. The hair conditioning composition of claim 1 wherein the cationic surfactant is an alkyl diquaternized ammonium salt selected from the group consisting of following (A1), (A2), and mixtures thereof:
(A1) an alkyl diquaternized ammonium salt having the formula: 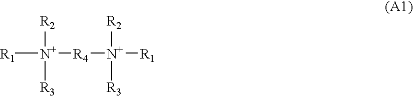
wherein R₁ represents straight or branched, saturated or unsaturated, and/or functionalized or non-functionalized C12-C40 alkyl chain, wherein the functionalized alkyl chain is that containing one or more functional groups selected from the group consisting of —OH, —COO—, —OCO—, —CONH—, —NHCO—, —O—, and —(R₅—O)ₙ— wherein R₅ is C2-4 alkylene and n is integer from 0 to 30; R₂ represents C1-3 alkyl, hydroxyalkyl, or —(R₅—O)ₙ—H; R₃ represents R₂ or H; and R₄ represents functionalized or non-functionalized C2-8 alkylene, wherein the functionalized alkylene is that containing one or more functional groups selected from the group consisting of —OH, —COO—, —OCO—, —CO—NH—, —O—, and —(R₅—O)ₙ—; and
(A2) an alkyl diquaternized ammonium salt having the formula: 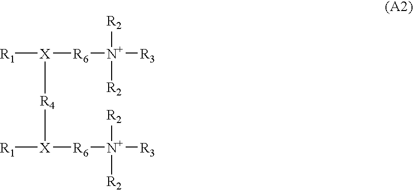
wherein R₁-R₄ represent the same structures as described above; X represents CH or N; and R₆ represents functionalized or non-functionalized C1-10 alkylene wherein the functionalized alkylene is that containing one or more functional groups selected from the group consisting of —OH, —COO—, —OCO—, —CONH—, —NHCO—, —O—, and —(R₅—O)ₙ—.

11. The hair conditioning composition comprising of claim 10 wherein the alkyl diquaternized ammonium salt cationic surfactant is selected from that having the formula (A1).

12. The hair conditioning composition comprising of claim 10 wherein the alkyl diquaternized ammonium salt cationic surfactant is selected from that having the formula (A1) wherein R₄ represents C2-8 alkylene functionalized with —OH.

13. The hair conditioning composition of claim 10 wherein the alkyl diquaternized ammonium salt cationic surfactant is selected from that having the formula (A2).

14. The hair conditioning composition of claim 1 wherein the composition is substantially free of other cationic surfactants, wherein such other cationic surfactants comprise mono-long alkyl quaternized ammonium salt in which the anion is not C1-C4 alkyl sulfate, tertiary amines, tertiary amine salts, and di-long alkyl quaternized ammonium salts.

15. The hair conditioning composition of claim 1 wherein the composition is substantially free of anionic surfactants and anionic polymers.

16. The hair conditioning composition of claim 1 wherein the weight ratio of the cationic surfactant and the high melting point fatty compound is within the range of from about 1:1 to about 1:4.

17. The hair conditioning composition of claim 8 wherein the total of the cationic surfactant and the high melting point fatty compound is present in the composition at a level of 7.5% or more.

18. The hair conditioning composition of claim 1 further comprising from about 0.1% to about 20% of a silicone compound.

19. The hair conditioning composition of claim 18 wherein the silicone compound is an aminosilicone having the general formula:
(R₁)ₐG_{3-z}-Si—(—OSiG₂)ₙ-(—OSiG_{b}(R₁)_{2-b})ₘ—O—SiG₃₋ₐ(R₁)ₐ
wherein G is hydrogen, phenyl, hydroxy, or C₁-C₈ alkyl; a is 0 or an integer having a value from 1 to 3; n is a number from 0 to 1,999; m is an integer from 0 to 1,999; the sum of n and m is a number from 1 to 2,000; a and m are not both 0; R₁ is a monovalent radical conforming to the general formula C_{q}H_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical; A⁻ is a halide ion.

20. The hair conditioning composition of claim 19 wherein the aminosilicone is mixed with a solvent having a low viscosity selected from the group consisting of non-polar, volatile hydrocarbons, volatile cyclic silicones, non-volatile linear silicones, and mixtures thereof.

21. The hair conditioning composition of claim 18 wherein the silicone compound is a mixture of (i) a first silicone having a viscosity of from about 100,000 mPa·s to about 30,000,000 mPa·s at 25° C. and (ii) a second silicone having a viscosity of from about 5 mPa·s to about 10,000 mPa·s at 25° C.

22. A method of conditioning hair, the method comprising following steps:
(i) after shampooing hair, applying to the hair an effective amount of the conditioning composition of claim 1 for conditioning the hair; and
(ii) then rinsing the hair.
